(19) United States
(12) Patent Application Publication
(10) Pub. No.: US 2008/0306392 A1
(43) Pub. Date: Dec. 11, 2008

(54) BLOOD VESSEL IMAGING APPARATUS

(75) Inventors: Hideki Satoguchi, Kawasaki (JP); Narutaka Nakao, Kawasaki (JP); Takahiro Aoki, Kawasaki (JP); Masahide Yaginuma, Sendai (JP)

Correspondence Address: GREER, BURNS & CRAIN, 300 S WACKER DR, 25TH FLOOR, CHICAGO, IL, 60606, US

(73) Assignee: FUJITSU LIMITED, Kawasaki-shi (JP)

(21) Appl. No.: 12/137,140

(22) Filed: Jun. 11, 2008

(30) Foreign Application Priority Data
Jun. 11, 2007 (JP) 2007-153848

(51) Int. Cl. A61B 5/02

(52) U.S. Cl. 600/479

(57) ABSTRACT

An imaging device used for a blood vessel imaging apparatus includes: a housing; an image sensor mounted in the housing; a control circuit board fixedly held in the housing and provided with the image sensor; an illumination circuit board disposed in the housing so as to be opposed to the control circuit board, and having an opening in a position opposed to the image sensor; a tubular light intercepting member disposed so as to cover the periphery of the image sensor and protrude through the opening; and an optical part attached to the tubular light intercepting member.

### CROSS-REFERENCE TO RELATED APPLICATIONS

This application is related to and claims priority to Japanese patent application no. 2007-153848 filed on Jun. 11, 2007 in the Japan Patent Office, and incorporated by reference herein.

### TECHNICAL FIELD

The present embodiment relates to a blood vessel imaging apparatus for visualizing a vein from the outside of the human body, and more specifically, to a video camera used for the blood vessel imaging apparatus.

Japanese Patent JP-8-510393 (JP '393) describes a blood vessel imaging apparatus that visualizes the position of a vein of the human body by using near infrared light. The blood vessel imaging apparatus is used for quickly and precisely performing an intravenous injection or an intravenous drip. This blood vessel imaging apparatus uses a characteristic of hemoglobin in blood absorbing near infrared light. The blood vessel imaging apparatus emits near infrared light, for example, to the back of a hand and detects the reflected light therefrom by an image sensor to thereby visualize the vein of the back of the hand. The blood vessel imaging apparatus then projects the vein image onto the back of the hand. This enables easy visual recognition of the position of the vein. If the position of the vein can be visually recognized with ease like this, the operator can quickly and reliably perform an intravenous injection or an intravenous drip.
[CITE THIS REFERENCE IN AN IDS]

The blood vessel imaging apparatus disclosed in JP '393 is comparatively large in size because it projects a vein image onto the human body. As a result, uses of this blood vessel imaging apparatus are limited. For example, when an intravenous injection is given to a premature baby nursed in an incubator equipped with oxygen supplementation, ventilation, infection prevention and the like, it is undesirable to use the blood vessel imaging apparatus disclosed in JP '393. This is because moving the baby out of the incubator to the blood vessel imaging apparatus involves risks of infection and the like.

Moreover, for example, when the position of a blood vessel in the abdomen is imaged during an abdominal operation, it is virtually impossible to use the blood vessel imaging apparatus of JP '393.

### SUMMARY

According to an aspect of an embodiment, an imaging device used for a blood vessel imaging apparatus is provided with: a housing serving as a casing of the imaging device; an image sensor mounted in the housing; a control circuit board fixedly held in the housing and operatively connected to the image sensor; an illumination circuit board disposed in the housing so as to be opposed to the control circuit board, and having an opening in a position opposed to the image sensor; a tubular light intercepting member disposed so as to cover a periphery of the image sensor and protrude through the opening; and an optical part attached to the tubular light intercepting member.

### BRIEF DESCRIPTION OF THE DRAWINGS

FIG. 1 is a perspective view of a blood vessel imaging apparatus according to the present embodiment;

FIG. 2 is a front view of the blood vessel imaging apparatus;

FIG. 3 is a perspective view of a video camera used for the blood vessel imaging apparatus;

FIG. 4 is a longitudinal cross-sectional view of the video camera;

FIG. 5 is a bottom view of the video camera;

FIG. 6 is a block diagram of the blood vessel imaging apparatus;

FIG. 7 is a block diagram showing a first modification of an LED drive circuit; and

FIG. 8 is a block diagram showing a second modification of the LED drive circuit.

### DETAILED DESCRIPTION OF THE EMBODIMENTS

An imaging device according to the present embodiment is an imaging device used for a blood vessel imaging apparatus. The apparatus has a housing serving as the casing of the imaging apparatus; a control circuit board fixedly held in the housing; an illumination circuit board disposed in the housing so as to be opposed to the control circuit board; an image sensor mounted on one surface of the control circuit board, that surface being opposed to the illumination circuit board; and an optical part attached to a tubular light intercepting member disposed on the surface of the control circuit board so as to cover the periphery of the image sensor and protrude through an opening formed in the illumination circuit board in a position opposed to the image sensor.

As described above, in the imaging device according to the present embodiment, the control circuit board and the illumination circuit board are disposed with a gap therebetween in the housing. The tubular light intercepting member is interposed therebetween, and the optical part is attached to an end of the tubular light intercepting member. In this manner, the overall size of the imaging device can be made small and compact.

The imaging device according to the present embodiment is provided with, as required, a visible light cut-off filter disposed in the housing so as to cover an end of the optical part and a plurality of illumination elements mounted so as to surround an end of the tubular light intercepting member on one surface of the illumination circuit board. That surface is opposed to the visible light cut-off filter.

Since the visible light cut-oft filter disposed in the housing so as to cover the end of the optical part and the plurality of illumination elements mounted so as to surround the end of the tubular light intercepting member on the surface of the illumination circuit board opposed to the visible light cut-off filter are provided, external noise light and illumination light from the illumination elements on the image sensor can be blocked by the tubular light intercepting member and the visible light cut-off filter, which enables sharp imaging by the image sensor.

The illumination elements can be light emitting diodes. If the illumination elements are light emitting diodes, the image sensor can be made thinner and more compact.

An LED drive circuit for driving the light emitting diodes by a pulse modulation method is mounted on the illumination circuit board. Since the LED drive circuit for driving the light emitting diodes is mounted on the illumination circuit board, the light emission of the light emitting diodes can be controlled under an illumination condition optimum for the object to be imaged, so that imaging by the image sensor can be more sharply performed.

The light emitting diodes are preferably more than one in number. The light emitting diodes are divided into a plurality of groups, and the plurality of groups can be driven by LED drive circuits independent of each other, respectively. Consequently, even when a failure or the like occurs in one group, this can be backed up by other groups, so that imaging can be performed with reliability.

In the imaging device according to the present embodiment, two of the light emitting diodes can be disposed so as to be opposed to each other in the direction of the diameter of the tubular light intercepting member. In this manner, the light emitted from the light emitting diodes to the object to be imaged can be uniformized as much as possible, so that imaging by the image sensor can be performed more accurately.

With multiple light emitting diodes, the light emitting diodes being divided into two groups, the light emitting diodes of the two groups can be alternately arranged in an annular shape. The diodes can be connected in series in each group, and the groups of the light emitting diodes can be driven by LED drive circuits independent of each other using currents of opposite directions, respectively. The imaging device according to the present embodiment, the light emitting diodes are more than one in number, the light emitting diodes are divided into two groups, the light emitting diodes of the two groups are alternately arranged in an annular shape, and the groups of the light emitting diodes are driven by LED drive circuits independent of each other by currents of opposite directions of the annular shape, respectively. As a result, the electromagnetic noise caused by the drive pulse applied to the light emitting diodes can be canceled out by the electromagnetic noise caused by the light emitting diodes of the groups. Consequently, the superimposition of noise on the imaging signal taken by the image sensor can be suppressed, so that a sharp image can be realized.

A blood vessel imaging apparatus according to the present embodiment is provided with: a housing serving as the casing of the blood vessel imaging apparatus; a control circuit board fixedly held in the housing; an illumination circuit board disposed in the housing so as to be opposed to the control circuit board with a gap in between; an image sensor mounted on a surface of the control circuit board opposed to the illumination circuit board; and an optical part attached to a tubular light intercepting member disposed on the surface of the control circuit board so as to cover the periphery of the image sensor and protrude through an opening formed in the illumination circuit board in a position opposed to the image sensor.

By disposing the control circuit board and the illumination circuit board with a gap therebetween in the housing, the tubular light intercepting member is interposed therebetween, and the optical part is attached to an end of the tubular light intercepting member, so that the overall size of the blood vessel imaging apparatus can be made small and compact.

### [Best Mode for Carrying out the Invention]

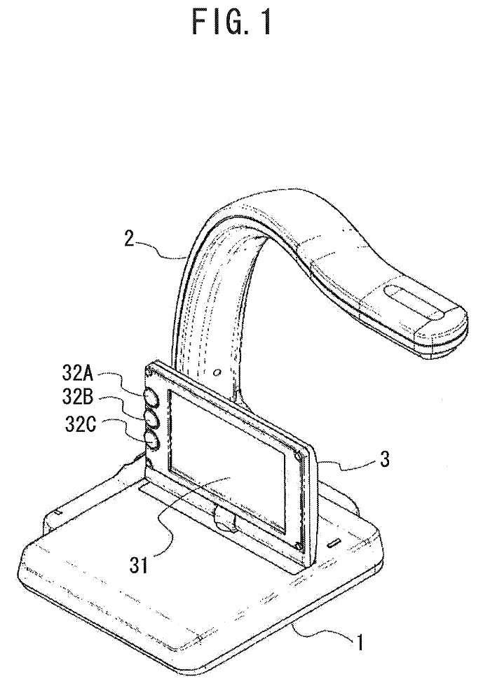

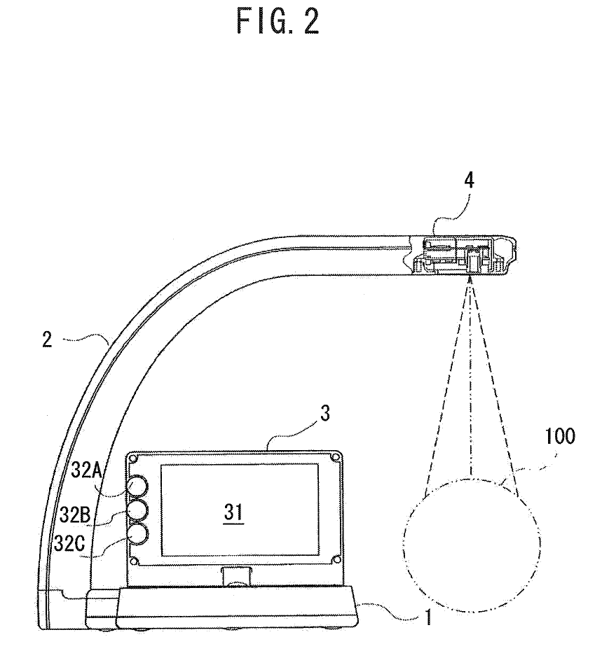

The outline of the structure of a blood vessel imaging apparatus according to an embodiment of the present embodiment will be described with reference to FIGS. 1 and 2. FIG. 1 is a perspective view of the blood vessel imaging apparatus. FIG. 2 is a partially cutaway front view of the blood vessel imaging apparatus of FIG. 1.

The blood vessel imaging apparatus according to the present embodiment is used for visualizing the position of the vein at the time of an intravenous injection or blood taking. As shown in FIGS. 1 and 2, the blood vessel imaging apparatus has a base 1 for placement on a table of medical equipment or the like. The blood vessel imaging apparatus has an arm 2 extending so as to protrude from the left side of the base 1 toward the right side thereof beyond the base 1. The blood vessel imaging apparatus has a liquid crystal display 3 mounted on the base 1 so as to be tiltable backward and forward. The liquid crystal display 3 has a rectangular liquid crystal (LCD) panel 31 having its lower side tiltably supported. The liquid crystal display 3 has three adjustment knobs 32A, 32B, and 32C on the left side of the LCD panel 31. Two of these three adjustment knobs 32A, 32B, and 32C, namely, the adjustment knobs 32A and 32B are used for adjusting the brightness and contrast of the LCD panel 31.

FIG. 2 shows the blood vessel imaging apparatus so as to be partially cutaway. The blood vessel imaging apparatus shown in FIG. 2 has a video camera 4 incorporated in an end portion of the arm 2. The video camera 4 images an object to be imaged 100 situated on the right side of the base 1. The video camera 4 is an imaging device.

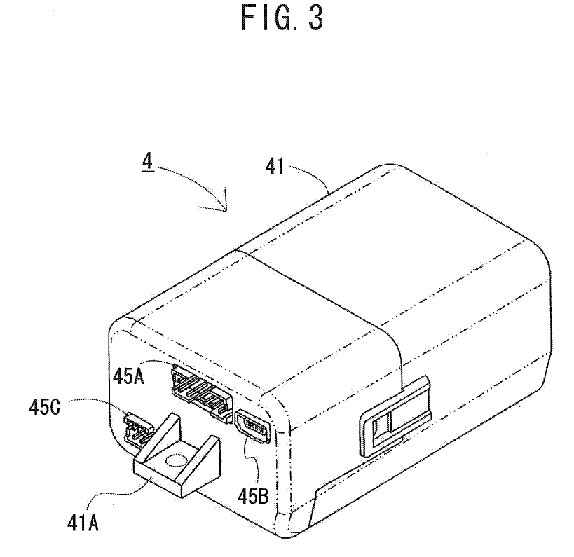

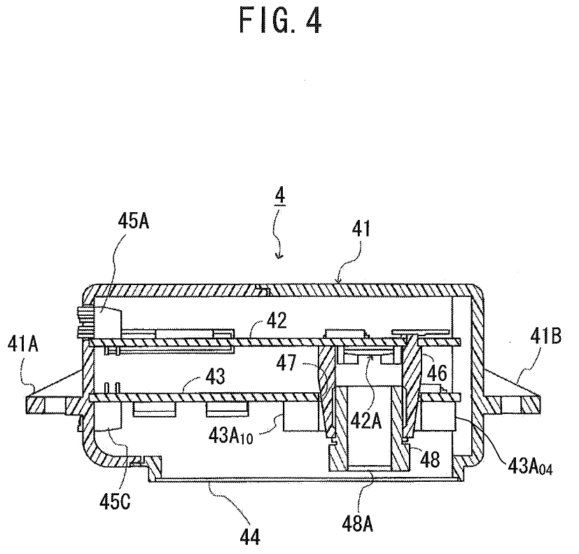

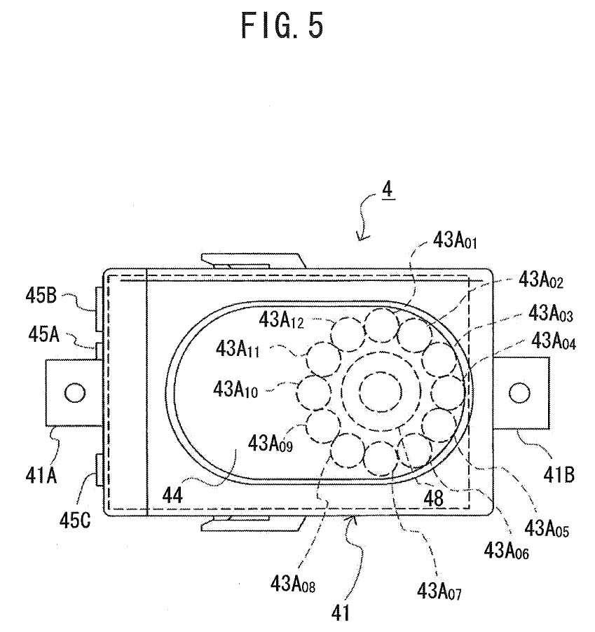

Next, the video camera 4 will be described with reference to FIGS. 3, 4, and 5. FIG. 3 is a perspective view of the video camera 4. FIG. 4 is a longitudinal cross-sectional view of the video camera 4. FIG. 5 is a bottom view of the video camera 4.

FIGS. 3 and 4 show the video camera 4. The casing of the video camera 4 shown in FIG. 4 is a housing 41. The housing 41 is made of a synthetic resin material or the like. The housing 41 is formed so that attachment pieces 41A and 41B integrally protrude from both end wall surfaces thereof. The attachment pieces 41A and 41B are used for securing the video camera 4 by screws. The video camera 4 is screwed in the end portion of the arm 2.

The video camera 4 has a control circuit board 42 and an illumination circuit board 43 fixedly held in the housing 41. The control circuit board 42 is disposed on the top side of the housing 41. The illumination circuit board 43 is disposed on the bottom side of the housing 41. The illumination circuit board 43 is disposed so as to be parallelly opposed to the control circuit board 42 at a predetermined distance therefrom. In the video camera 4, the housing 41 has a visible light cut-off filter 44. The visible light cut-off filter 44 is disposed so as to cover the opening provided at the bottom. That is, the visible light cut-off filter 44 covers an end of an optical part 48. The visible light cut-off filter 44 is provided on the housing 41.

FIG. 3 shows the housing 41. The housing 41 has connectors 45A, 45B, and 45C on one end surface thereof. The connector 45A is mounted on the control circuit board 42 (see FIG. 4). The connector 45A is connected to the liquid crystal display 3 through a signal cord extending in the arm 2 (see FIGS. 1 and 2). The connector 45B can be a USB (universal serial bus) connector. The connector 45B is also mounted on the control circuit board 42. The connector 45C is mounted on the illumination circuit board 43 (see FIG. 4). The illumination circuit board 43 is connected to a DC power supply circuit 11 through a power supply cord extending in the arm 2. The DC power supply circuit 11 is provided in the base 1 (see FIGS. 1 and 2). The DC power supply circuit 11 is connected to the commercial power supply.

FIG. 4 shows the control circuit board 42. An image sensor 42A is mounted on one surface, that is, on the surface, opposed to the illumination circuit board 43, of the control circuit board 42. As the image sensor 42A, for example, a CMOS (complementary metal oxide semiconductor) image sensor or a CCD (charge coupled device) image sensor is used. On the above-mentioned surface of the control circuit board 42, a tubular light intercepting member 46 is attached so as to cover the periphery of the image sensor 42A. An end of the tubular light intercepting member 46 passes through a circular opening 47 of the illumination circuit board 43. The end of the tubular light intercepting member 46 is set so as to protrudes through and out of the circular opening 47.

On the control circuit board 42, various electronic parts are mounted as well as the image sensor 42A. Primary ones of these electronic parts will be described later in detail.

FIG. 4 shows the tubular light intercepting member 46. The optical part 48 is attached to the inside of the end of the tubular light intercepting member 46. The optical part 48 incorporates an image forming optical system 48A. That is, the tubular light intercepting member 46 not only prevents the incidence of noise light on the image sensor 42A but also functions as an optical part holder for holding the optical part 48. In the present embodiment, the image forming optical system 48A has a fixed focus, and forms an image of the object to be imaged 100 approximately 30 cm ahead, on the tight receiving surface of the image sensor 42A.

FIGS. 4 and 5 show the illumination circuit board 43. One surface of the illumination circuit board 43 is opposed to the visible light cut-off filter 44. On the surface opposed to the visible light cut-off filter 44, twelve light emitting diodes 43A01 to 43A12 are mounted so as to surround the end of the tubular light intercepting member 46. The twelve light emitting diodes are illumination elements.

On the illumination circuit board 431 various electronic parts are mounted as well as the light emitting diodes 43A01 to 43A12. Primary ones of these electronic parts will be described later in detail.

In the video camera 4 according to the present embodiment, the control circuit board 42 and the illumination circuit board 43 are disposed with a predetermined gap therebetween in the housing 41, the tubular light intercepting member 46 surrounding the image sensor 42A is disposed in the predetermined gap, and the optical part 48 is attached to the tubular light intercepting member 46, whereby the video camera 4 can be made small in size and compact.

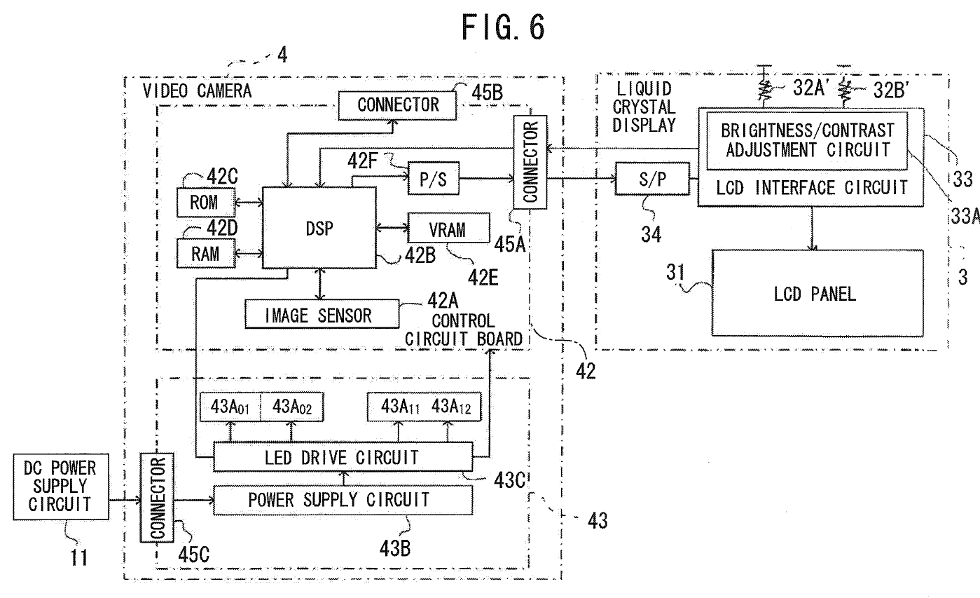

FIG. 6 is a block diagram. The block diagram of FIG. 6 illustrates the video camera and the liquid crystal display of the blood vessel imaging apparatus shown in FIGS. 1 to 5.

FIG. 6 shows the liquid crystal display 3. The liquid crystal display 3 has, in addition to the LCD panel 31, an LCD interface circuit 33 for driving the LCD panel 31. The LCD interface circuit 33 includes a brightness/contrast adjustment circuit 33A. The adjustment knobs 32A and 32B operate variable resistors 32A′ and 32B′. The resistance values of the variable resistors 32A′ and 32B′ are changed by adjustment of the adjustment knobs 32A and 32B, respectively. The variable resistors 32A′ and 32B′ are connected to the brightness/contrast adjustment circuit 33A.

When the operator operates the brightness adjustment knob 32A, the resistance value of the variable resistor 32A′ is changed. The brightness/contrast adjustment circuit 33A outputs, to the video camera 4, a brightness adjustment signal based on the resistance value of the variable resistor 32A′. Thereby, the liquid crystal display 3 displays a brightness-adjusted image.

On the other hand, when the operator operates the contrast adjustment knob 32B, the resistance value of the variable resistor 32B′ is changed. The brightness/contrast adjustment circuit 33A outputs a contrast adjustment signal based on the resistance value of the variable resistance 32B′. The contrast adjustment signal is outputted to the LCD panel 31 through the LCD interface circuit 33. Thereby, the liquid crystal display 3 adjusts the contrast of the LCD panel 31.

The liquid crystal display 3 also has an S/P (serial-to-parallel) converter 34 as described later. The S/P converter 34 converts the serial video signal outputted from the video camera 4, into a parallel video signal. This parallel video signal is inputted to the LCD interface circuit 33. After having undergone appropriate processing, the parallel video signal is outputted to the LCD panel 31. Thereby, an image based on the parallel video signal is displayed on the LCD panel 31.

As shown in FIG. 6, on the control circuit board 42, primary electronic parts are mounted as well as the image sensor 42A. The primary electronic parts include a DSP (digital signal processor) 42B, a ROM (read only memory) 42C, and a RAM (random access memory) 42D. The DSP 42B functions as an image processor. The ROM 42C stores various programs, constants, and the like. The ROM 42C is a read only memory. The RAM 42D stores temporary data while the DSP 42B is operating. The RAM 42D is a readable and writable memory.

A VRAM (video random access memory) 42E is also mounted on the control circuit board 42. The VRAM 42E is used for processing the image signal successively obtained from the image sensor 42A. The DSP 42B produces a parallel video signal based on the image signal processed by the VRAM 42E. Further, a P/S (parallel-to-serial) converter 42F is mounted on the control circuit board 42. The parallel video signal produced by the DSP 42B is converted into a serial video signal by the P/S converter 42F. This serial video signal is sent to the S/P converter 34 of the liquid crystal display 3 through the connector 45A.

The brightness adjustment signal is outputted from the brightness/contrast adjustment circuit 33A of the liquid crystal display 3. The brightness adjustment signal is sent to the DSP 42B through the connector 45A. The USB connector 45B is connected to the DSP 42B. The USB connector 45B transmits and receives signals to and from an external apparatus (not shown).

FIG. 6 is a block diagram of the blood vessel imaging apparatus. The light emitting diodes 43A01 to 43A12 are mounted on the illumination circuit board 43. On the illumination circuit board 43, a power supply circuit 43B and an LED drive circuit 43C are mounted as primary electronic components. The power supply circuit 43B is connected to the DC power supply circuit 11 provided in the base 1 (see FIGS. 1 and 2), through the connector 45C. The DC power supply circuit 11 is connected to the commercial power supply. The LED drive circuit 43C is supplied with power by the power supply circuit 43B. Thereby, the light emitting diodes 43A01 to 43A12 emit light. The power supply circuit 43B also supplies power to the electronic parts 42A to 42F and the like mounted on the control circuit board 42.

The LED drive circuit 43C operates on the pulse modulation method. According to the pulse modulation method, the brightness of the light emitting diodes 43A01 to 43A12 is adjusted. That is, the LED drive circuit 43C outputs a drive pulse of a predetermined frequency to each of the light emitting diodes 43A01 to 43A12. The LED drive circuit 43C adjusts the duty ratio of the drive pulse. Thereby, the brightness of the illumination light of each of the light emitting diodes 43A01 to 43A12 is controlled. Describing this in detail, the duty ratio of the drive pulse is controlled based on the brightness adjustment signal outputted from the brightness/contrast adjustment circuit 33A of the liquid crystal display 3. Thereby, the brightness of the illumination LEDs 43A01 to 43A12 is adjusted.

That is, when the subject image on the LCD panel 31 is too dark, adjustment is made by the brightness adjustment knob 32A so that the image becomes brighter. For example, the resistance value of the variable resistor 32A′ is increased by the brightness adjustment knob 32A. By doing this, the duty ratio of the drive pulse of the illumination LEDs 43A01 to 43A12 is increased. Thereby, the illumination light amount of the illumination LEDs 43A01 to 43A12 is increased. Consequently, the amount of illumination light emitted to the subject is increased, whereby the image on the LCD panel 31 becomes brighter. On the contrary, when the image on the LCD panel 31 is too bright, adjustment is made by the brightness adjustment knob 32A so that the image becomes darker. For example, the resistance value of the variable resistor 32A′ is decreased by the brightness adjustment knob 32A. By doing this, the duty ratio of the drive pulse is decreased. Consequently, the image on the LCD panel 31 becomes darker.

The operation of the blood vessel imaging apparatus will be described based on the block diagram of FIG. 6. For example, when an intravenous injection is given to an arm of the human body or blood is taken from the arm, the joint part of the arm is placed under the video camera 4 so that the inside thereof faces toward the video camera 4 (see FIGS. 1 and 2). At this time, the light emitted from the light emitting diodes 43A01 to 43A12 passes through the visible light cut-off filter 44. The light emitted from the light emitting diodes 43A01 to 43A12 illuminates the arm. That is, the arm is illuminated by near infrared light because of the visible light cut-off filter 44 (see FIGS. 4 and 5). The reflected light of the near infrared light is detected by the image sensor 42A. At this time, the vein image is visualized as a dark area. This is because the hemoglobin in the vein absorbs near infrared light as mentioned above.

From the image sensor 42A, for example, twenty frames of image signals are read out per second. These twenty frames of image signals are successively processed as appropriate by the DSP 42B. The image signals processed by the DSP 42B are stored in the VRAM 42E. The DSP 42B produces the video signal based on the image signals stored in the VRAM 42E. The video signal is outputted as a parallel video signal to the P/S converter 42F, where the parallel video signal is converted into a serial video signal. The serial video signal is then sent to the S/P converter 34 of the liquid crystal display 3.

The S/P converter 34 of the liquid crystal display 3 returns the serial video signal to a parallel video signal. This parallel video signal is outputted to the LCD interface circuit 33. Thereby, an image of the arm is displayed on the LCD panel 31. At this time, a vein image is projected on the inside of the arm as a dark area. Consequently, the injection needle can be quickly and precisely inserted into a predetermined position of the vein.

The above-described blood vessel imaging apparatus is used for giving an intravenous injection to an arm or the back of a hand, or taking blood. For other uses, the arm 2 can be removed according to the use. In this case, the video camera 4 and the liquid crystal display 3 are connected together by the signal cord and the power supply cord. That is, this blood vessel imaging apparatus can be operated with the video camera 4 held in a hand.

The hand-holdable video camera 4 can be used, for example, when an intravenous injection is given to or blood is taken from a premature baby in an incubator. Specifically, the vein of an arm of the premature baby is projected on the LCD panel 31 with the video camera 4 placed on the roof part (transparent glass) of the incubator, whereby an intravenous injection or blood taking can be quickly and precisely performed while the premature baby is in the incubator.

Moreover, the hand-holdable video camera 4 can be used, for example, for abdominal operations. That is, by projecting an artery in the abdomen on the LCD panel 31, the operator can easily and precisely grasp the position of the artery, so that the abdominal operation can be safely performed.

In the above-described video camera 4, although the light emitting diodes 43A01 to 43A12 have a comparatively long life, failure readily occurs in the LED drive circuit 43C. For this reason, there can be cases where the light emitting diodes 43A01 to 43A12 do not emit light because of a failure of the LED drive circuit 43C although the light emitting diodes 43A01 to 43A12 are not dead.

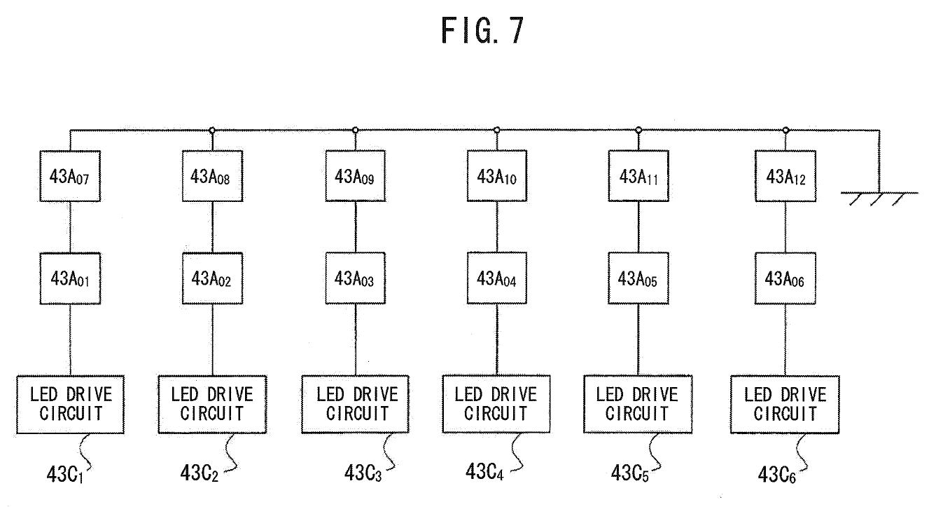

To avoid this, for example, as shown in FIG. 7, the LED drive circuit 43C is divided into six LED drive circuits 43C1 to 43C6, and each of the LED drive circuits 43C1 to 43C6 drives two of the twelve light emitting diodes 43A01 to 43A12.

FIG. 7 is a block diagram of the LED drive circuit. The LED drive circuit 43C1 drives the light emitting diodes 43A01 and 43A07. The LED drive circuit 43C2 drives the light emitting diodes 43A02 and 43A08. The LED drive circuit 43C3 drives the light emitting diodes 43A03 and 43A09. The LED drive circuit 43C4 drives the light emitting diodes 43A04 and 43A10. The LED drive circuit 43C5 drives the light emitting diodes 43A05 and 43A11. The LED drive circuit 43C6 drives the light emitting diodes 43A06 and 43A12.

Even when a failure occurs in one or two of the six LED drive circuits 43C1 to 43C6, only two or four of the twelve light emitting diodes 43A01 to 43A12 do not emit light. However, by increasing the light emission intensity of the remaining light emitting diodes, an image of the desired brightness can be obtained on the LCD panel 31.

In the example shown in FIG. 7, when a failure occurs in any of the LED drive circuits 43C1 to 43C6, illumination nonuniformity can be avoided as much as possible. For this purpose, the two light emitting diodes driven by one LED drive circuit 43C1, 43C2, 43C3, 43C4, 43C5, or 43C6 are disposed on a diagonal line (see FIG. 5). The two light emitting diodes may be disposed with one or two other light emitting diodes in between.

On the other hand, when the pulse modulation method is adopted for the light emission of the light emitting diodes 43A01 to 43A12, electromagnetic noise is caused on the rising edge or the falling edge of the drive pulse. There are cases where this electromagnetic noise is mixed in the video signal to disturb the image on the LCD panel 31.

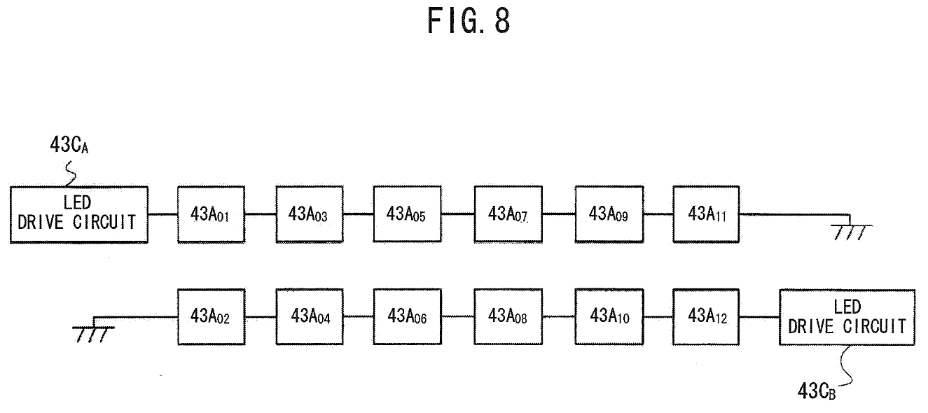

FIG. 8 shows an example to avoid this. The light emitting diodes 43A01 to 43A12 are divided into a first group and a second group. Every other light emitting diode, namely, six light emitting diodes 43A01, 43A03, 43A05, 43A07, 43A09, and 43A11 belong to the first group. The other light emitting diodes, namely, the six light emitting diodes 43A02, 43A04, 43A06, 43A08, 43A10, and 43A12 belong to the second group. The light emitting diodes of the first group and the light emitting diodes of the second group are driven by LED drive circuits 43CA and 43CB independent of each other, respectively. The light emitting diodes of the first group and the light emitting diodes of the second group are driven by currents of opposite directions.

That is, the wiring of the light emitting diodes of the first group and the wiring of the light emitting diodes of the second group are disposed around the light emitting diodes 43A01 to 43A12. The wiring of the light emitting diodes of the first group and the wiring of the light emitting diodes of the second group are disposed adjacent to each other. The wiring of the light emitting diodes of the first group and the wiring of the light emitting diodes of the second group are disposed on the illumination circuit board 43. In this manner, the electromagnetic noises caused on both wirings cancel each other out. Consequently, the mixture of electromagnetic noise in the video signal can be prevented.

What is claimed is:

1. An imaging device used for a blood vessel imaging apparatus, comprising:
a housing serving as a casing of the imaging device;
an image sensor mounted in the housing;
a control circuit board fixedly held in the housing and provided with the image sensor;
an illumination circuit board disposed in the housing so as to be opposed to the control circuit board, and having an opening in a position opposed to the image sensor; and
an optical part attached to a tubular light intercepting member disposed so as to cover a periphery of the image sensor and protrude through the opening.

2. The imaging device according to claim 1, comprising:
a visible light cut-off filter disposed in the housing so as to cover an end of the optical part; and
a plurality of illumination elements mounted so as to surround an end of the tubular light intercepting member on a surface of the illumination circuit board opposed to the visible light cut-off filter.

3. The imaging device according to claim 2,
wherein the illumination elements are light emitting diodes.

4. The imaging device according to claim 3,
wherein an LED drive circuit for driving the light emitting diodes by a pulse modulation method is mounted on the illumination circuit board.

5. The imaging device according to claim 4,
wherein the light emitting diodes are more than one in number, the more than one light emitting diodes are divided into a plurality of groups, and the plurality of groups of the light emitting diodes are driven by LED drive circuits independent of each other, respectively.

6. The imaging device according to claim 5,
wherein the groups of the light emitting diodes are disposed so as to be opposed to each other with the tubular light intercepting member in between.

7. The imaging device according to claim 4,
wherein the light emitting diodes are more than one in number, the light emitting diodes are divided into two groups, the light emitting diodes of the two groups are alternately arranged in an annular shape, and the groups of the light emitting diodes are driven by LED drive circuits independent of each other by currents of opposite directions of the annular shape, respectively.

8. A blood vessel imaging apparatus comprising:
an imaging device;
a housing serving as a casing of the imaging device;
an image sensor mounted in the housing;
a control circuit board fixedly held in the housing and provided with the image sensor;
an illumination circuit board disposed in the housing so as to be opposed to the control circuit board, and having an opening in a position opposed to the image sensor; and
an optical part attached to a tubular light intercepting member disposed so as to cover a periphery of the image sensor and protrude through the opening.
